SEQUENCE LISTING (12 sequences)

SEQ ID NO: 1 - protein, 316 aa, Pseudomonas aeruginosa
MNPNFLDFEQPIADLQAKIEELRLVGNDNALNISDEISRLQDKSKALTENIFGNLSSWQIAQLARHPKRPYTLDYIGYLFSDFEELHGDRHFADDPAIVGGVARLDGSPVMVIGHQKGREVREKVRRNFGMPRPEGYRKACRLMEMAERFKMPILTFIDTPGAYPGIDAEERGQSEAIAWNLRVMARLKTPIIATVIGEGGSGGALAIGVCDQLNMLQYSTYSVISPEGCASILWKTAEKAPEAAEAMGITAERLKGLGIVDKVIDEPLGGAHRDPASMAESIRGELLAQLKMLQGLEMGELLERRYDRLMSYGAP

SEQ ID NO: 2 - DNA, 951 bp, Pseudomonas aeruginosa
atgaacccgaactttcttgatttcgaacagccgatcgccgacctgcaagccaagatcgaagagctgcgcctggtgggcaacgacaatgcgctgaacatcagcgacgaaatctcgcgtctgcaggacaagagcaaggcgctcaccgaaaacatcttcggcaatctgtccagttggcagatcgcccagctcgcgcgccatcccaagcgtccctataccctcgactacatcggctacctgttcagcgatttcgaggaactgcacggcgaccggcatttcgccgacgacccggcgatcgtcggcggcgttgcccgcctcgacggttccccggtgatggtcatcggccaccagaagggccgcgaagtccgtgagaaggtccggcgcaacttcggcatgccgcgtccggaaggctatcgcaaggcctgccgcctgatggaaatggccgaacgcttcaagatgccgatcctcaccttcatcgacacgcccggcgcctacccggggatcgatgccgaggaacgcggccagagcgaggcgatcgcctggaacctgcgggtgatggcgcgactgaagacgccgatcatcgccaccgtgatcggcgagggcggttccggcggcgcgctggccatcggtgtctgcgaccagttgaacatgctgcaatactccacctattcggtgatctcgccggaaggctgcgcctccatcctctggaagaccgccgagaaggcgccggaagccgccgaggccatgggcatcaccgccgagcgcctgaaaggcctgggcatcgtcgacaaggtcatcgacgaaccgctgggcggcgcccatcgcgatccggcgagcatggccgaatcgatccgtggcgaactgctggcgcaactgaagatgctccagggcctggaaatgggtgagttgctggagcgtcgttacgaccgcctgatgagctacggcgcgccgtaa

SEQ ID NO: 3 - protein, 312 aa, Pseudomonas aeruginosa
MSASLAFVFPGQGSQSLGMLAELGAQQALVRDTFAEASEALGYDLWALVQNGPEERLNQTDKTQPAILTVSIALWRLWLAEGGARPAFVAGHSLGEYSALVAAESLAFADAVKLVERRGQLMQQAVPAGQGGMAAILGLEDADVLAACAEAAQGEVVSAVNFNAPGQVVIAGAAAAVERAIEACKARGAKRAVALPVSVPSHCELMRPAAEQFAASVESLQWQAPKISLVQNVSAAVPADLDTLRRDLLAQLYSPVRWVESIQLLAEKGVTELVECGPGKVLAGLNRRCAKGINTHGLDGVEAFAATRAALA

SEQ ID NO: 4 - DNA, 939 bp, Pseudomonas aeruginosa
atgtctgcatccctcgcattcgtcttccctggccagggttcgcaatccctcggcatgctggccgagctgggcgcccagcaggcgctggtgcgcgataccttcgccgaggcctccgaggcgctcggttacgacctttgggcgctggtccagaatggtcctgaagagcgcctgaaccagaccgacaagacccagccggccatccttacggtttcgatcgcgctctggcgcctctggctggccgagggcggtgcgcgcccggcgttcgtcgccgggcacagcctgggcgaatattccgcgctggtcgcggccgaaagcctggcgttcgccgatgcggtcaagctggtcgagcgtaggggccaactgatgcagcaggcggttccggcggggcagggcggcatggccgcgatccttggcctggaagacgccgatgtattggcggcctgtgccgaggcggcccagggcgaggtggtcagcgcggtcaacttcaacgcgccggggcaggtagtgatcgccggtgccgcggctgccgttgagcgtgccatcgaggcatgcaaggcacgcggcgccaagcgcgcggtggcgttgccagtcagcgtgccgtcgcattgcgaactgatgcgtccggccgccgagcagttcgccgcctcggtcgaaagcctgcagtggcaggcgccgaagatttcgctggtgcagaacgtcagcgccgccgtgccggctgatctcgatacgctgcgccgcgacctgctggcacagctgtacagcccggttcgctgggtggagagcatccagctgctggcggaaaagggcgtcaccgagctggtcgagtgcgggccgggcaaggtcctggcaggcctcaacaggcgctgcgcgaagggcatcaatacccatggcctggatggcgtcgaggcgttcgccgccacgcgcgccgccctggcctga

SEQ ID NO: 5 - protein, 254 aa, Streptococcus pyogenes
MPYKGYLIDLDGTIYQGKNRIPAGERFIKRLQEKGIPYLLVTNNTTRTPAMVQSMLANQFNVETGIETIYTATMATVDYMNDMNRGKTAYVIGETGLKSAIAAAGYVEELENPAYVVVGLDSQVTYEMLAIATLAIQKGALFIGTNPDLNIPTERGLMPGAGALNALLEAATRVKPVFIGKPNAIIMNKSLEVLGIQRSEAVMVGDNYLTDIMAGIQNDIATILVTTGFTRPEEVPTLPIQPDHVLSSLDEWRL

SEQ ID NO: 6 - DNA, 753 bp, Streptococcus pyogenes
atgggattaagttatcaggaggagttgacacttccttttgaattatgtgatgtcaaatcagatataaaattgccccttttattagactattgtttgatggtttctggtagacagtctgcgcaattaggacgaagtaacaacaaccttttagtcgattacaagcttgtttggattgtaacggattatgagatcactattcatcgcttgccacattttcaagaaaccatcaccattgaaacaaaagccctttcctataataaatttttttgttatcgccaattttatatttatgatcaagaggggggtcttttagtggatatcttagcctattttgctttgttaaacccagatacgcgaaaagtggcaactattccagaagatttagtagcgccttttaagactgattttgttaaaaagttataccgtgttcctaaaatgcctcttttagaacaatcaattgatcgtgattattatgtgcgttattttgatattgatatgaatggtcatgtcaacaacagtaaatatttagattggatgtatgatgtgttggggtgtgcgtttttaaaaacgcatcagcctcttaagatgactttgaaatatgttaaagaagtctcaccaggcggtcaaattacttccagttaccatttggaccaattaaattcttaccatcaaatcacctcagatgggcagctgaatgcccaagccatgattgaatggcgagcgattaaacaaacagaaagcgagatagactag

SEQ ID NO: 7 - DNA, 27 bp, Artificial Sequence
  forward primer for accA
tctagacgacggaagcctatgaacccg

SEQ ID NO: 8 - DNA, 27 bp, Artificial Sequence
  reverse primer for accA
ggatccttacggcgcgccgtagctcat

SEQ ID NO: 9 - DNA, 28 bp, Artificial Sequence
  forward primer for fabD
ggtacccaagggacctattcaatgtctg

SEQ ID NO: 10 - DNA, 29 bp, Artificial Sequence
  reverse primer for fabD
gagctcttctctcctttctctctctcagg

SEQ ID NO: 11 - DNA, 30 bp, Artificial Sequence
  forward primer for E.C 3.1.2.14
gagctcggagagtattatgggattaagtta

SEQ ID NO: 12 - DNA, 29 bp, Artificial Sequence
  reverse primer for E.C 3.1.2.14
gaattcctagtctatctcgctttctgttt

(19) United States
(12) Patent Application Publication
(10) Pub. No.: US 2013/0045515 A1
(43) Pub. Date: Feb. 21, 2013

(54) HETEROGENEOUS E. COLI FOR IMPROVING FATTY ACID CONTENT USING FATTY ACID BIOSYNTHESIS AND PREPARATION METHOD THEREOF

(71) Applicants: Jin-Won Lee, Seoul (KR); Sun-Hee Lee, Seoul (KR); Eun-Young Jeon, Seoul (KR)

(72) Inventors: Jin-Won Lee, Seoul (KR); Sun-Hee Lee, Seoul (KR); Eun-Young Jeon, Seoul (KR)

(73) Assignee: INDUSTRY-UNIVERSITY COOPERATION FOUNDATION SOGANG UNIVERSITY, Seoul (KR)

(21) Appl. No.: 13/642,131

(22) PCT Filed: Mar. 8, 2011
(86) PCT No.: PCT/KR11/01598

§ 371 Date: Oct. 19, 2012

(30) Foreign Application Priority Data
Apr. 22, 2010 (KR) 10-2010-0037546
Jan. 10, 2011 (KR) 10-2011-0002246

(51) Int. Cl. C12P 7/64; C12N 15/70; C12N 1/21

(52) U.S. Cl. 435/134; 435/252.33; 435/471

(57) ABSTRACT

Disclosed are novel E. coli strains, capable of producing fatty acids in high yield, transformed with genes coding for enzymes involved in the fatty acid biosynthesis pathway, and a method for preparing the same. The novel strains show excellent fatty acid production activity not only because their genetic stability is high, but also because they supply the basic materials through a two-way system.

### TECHNICAL FIELD

The present invention relates to a novel Escherichia coli strain capable of overexpressing enzymes involved in the fatty acid biosynthesis pathway, and a method for preparing the same.

### BACKGROUND ART

The burning of fossil fuels produces a tremendous amount of greenhouse gases and wastes that contribute to global warming, raising a serious environmental crisis to the mankind. Given this situation, there is a pressing need for the development of new, environmentally friendly bioprocesses using biomass as a fuel, as an alternative to the chemical processes based on fossil fuels, thus minimizing the production of detrimental wastes and the consumption of non-renewable energy. In recent years, there has been a great increase in interest in various bioenergy sources including bioethanol, biodiesel, biogas, and butanol. Some kinds of the bioenergy sources can be used as fuels for electricity production or transportation, but have drawbacks in their applicability and production. Now, attention is turning to renewable hydrocarbon compounds, with the concomitant increase of interest in recombinant strains capable of producing long chain fatty acids as products.

Pseudomonas aeruginosa was first obtained in pure culture by Gessard in 1882 from cutaneous wounds which had a blue green discolouration. This bacterial species is widely found in nature and frequently isolated from pus, phlegm, excreta, urine, bile, uterine secretions, blood, and spinal fluid. Pseudomonas aeruginosa is a Gram-negative, rod-shaped bacterium, and forms a mucous layer composed of extracellular polysaccharides, similar to capsules. This bacterium has great adaptability in any environment, with very simple auxotrophic requirements for growth. Streptococcus pyogenes is a spherical, Gram-positive bacterium that is the cause of many important human diseases, including pneumonia, pharyngitis, acute nephritis, and toxic shock syndrome.

A fatty acid is a monovalent carboxylic acid (—COOH) with a long hydrocarbon chain. Fatty acids are named as they are produced by the hydrolysis of lipids. In the backbone of fatty acid, hydrogen atoms are linked to each carbon atom, with a carboxyl group at one end. Fatty acids are degraded or synthesized in vivo through the fatty acid cycle. Most naturally occurring fatty acids have a chain of an even number of carbon atoms because two-carbon atom groups are cleaved from or added to the hydrocarbon chain of the fatty acid.

In microorganisms, the biosynthesis of fatty acids from sugar starts with acetyl-CoA. Then, the chain is lengthened as a two-carbon atom unit per cycle is added to the growing hydrocarbon chain. In the cytoplasm, acetyl-CoA is carboxylated into malonyl-CoA that plays as an important mediator in fatty acid biosynthesis. This irreversible carboxylation is catalyzed by acetyl-CoA carboxylase. The enzyme is composed of three enzymatic subunits and requires biotin and Mn²⁺ as a cofactor with the supply of ATP during the carboxylation. Two of the three carbon atoms in the malonyl moiety of malonyl-CoA are added to the growing fatty acid chain per cycle of the biosynthesis. Like the production of malonyl-CoA, this reaction is carried out in the cytoplasm by a multi-enzyme protein which is not bound to the membrane. This multi-enzyme protein composed of individual functional enzymes is named fatty acid synthase. The acyl carrier protein (ACP) is an important component of the fatty acid synthase, with the growing chain bound thereto during synthesis.

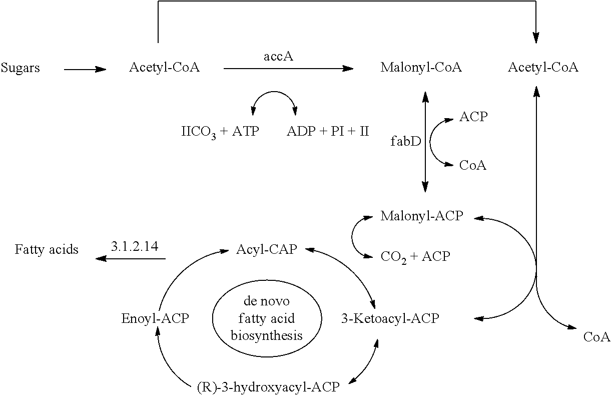

Incessant trials and efforts have been made to overexpress fatty acids in microbes using microbial fatty acid metabolism pathways. Information about the metabolism of E. coli is much more abundant, compared to other microbial organisms. In fact, E. coli is widely used for the production of recombinant proteins as all of its genes are revealed and analyzed. However, not much research has been conducted for the effect of the expression of exogenous genes in E. coli.

There are many studies on an improvement in the production of fatty acids by introduction of plant genes in E. coli, but genes from other microbial species were not much studied for improving fatty acid production, like the present invention.

E. coli is easy to manipulate and grows well and its metabolism pathways can be readily controlled while Pseudomonas aeroginosa is known to have a lipid content about twice as much as that of E. coli. The present inventors introduced a gene from Pseudomonas, which is rich in lipid including fatty acid, and a gene from a Gram-positive species, which is novel to both E. coli and Pseudomonas, into E. coli, so as to produce fatty acids at a greater efficiency.

In the present invention, gene manipulation was made, on the basis of the complete understanding of biological metabolism networks, to create a novel recombinant species which can produce a desired metabolite with high efficiency in an early step of the fatty acid biosynthesis pathway.

This application may reference various publications by author, citation, [symbol] by patent number, including without limitation, articles, presentations, and United States patents. The disclosures of each of any such references in their entireties are hereby incorporated by reference into this application.

### DISCLOSURE

### Technical Problem

Leading to the present invention, intensive and thorough research into the stable and effective production of a fatty acid biosynthesis metabolite by gene manipulation, conducted by the present inventors, resulted in the finding that when transformed with an accA gene, a fabD gene, both from Pseudomonas aeruginosa, and/or a 3.1.2.14 gene from Streptococcus pyogenes, E. coli can overexpress the enzymes involved in the fatty acid biosynthesis pathway to activate the pathway, thus producing the metabolite of interest at high yield.

It is therefore an object of the present invention to provide an E. coli transformant in which enzymes involved in the fatty acid biosynthesis pathway are overexpressed so as to producing a fatty acid at high yield.

It is another object of the present invention to provide a method for preparing the E. coli transformant.

It is a further object of the present invention to provide a method for the biosynthesis of a fatty acid using the E. coli transformant.

It is still a further object of the present invention to provide the use of the E. coli transformant in the biosynthesis of a fatty acid.

The above and other objects, features and other advantages of the present invention will be more clearly understood from the following detailed description and claims taken in conjunction with the accompanying drawings

### Technical Solution

Leading to the present invention, intensive and thorough research into the stable and effective production of a fatty acid biosynthesis metabolite by gene manipulation, conducted by the present inventors, resulted in the finding that when transformed with an accA gene, a fabD gene, both from Pseudomonas aeruginosa, and/or a 3.1.2.14 gene from Streptococcus pyogenes, E. coli can overexpress the enzymes involved in the fatty acid biosynthesis pathway to activate the pathway, thus producing the metabolite of interest in high yield.

In accordance with an aspect thereof, the present invention addresses an E. coli species capable of producing a fatty acid in high yield, co-transformed with an expression vector carrying a gene selected from the group consisting of: a nucleotide sequence coding for acetyl-CoA carboxylase carboxytransferase subunit alpha, a nucleotide sequence coding for malonyl-CoA-[acyl-carrier protein] transacylase, a nucleotide sequence coding for acyl-acyl carrier protein thioesterase, and a combination thereof.

As used herein, the term “acetyl-CoA carboxylase carboxytransferase” means the enzyme that catalyzes the carboxylation of acetyl-CoA to malonyl-CoA and forms a tetramer composed of two alpha and two beta subunits. One of the subunits corresponds to the acetyl-CoA carboxylase carboxytransferase subunit alpha, encoded by the accA gene.

Preferably, the acetyl-CoA carboxylase carboxytransferase subunit alpha expressed from the expression vector in accordance with the present invention is derived from Pseudomonas aeruginosa. More preferably, the acetyl-CoA carboxylase carboxytransferase subunit alpha has the amino acid sequence of SEQ ID NO: 1, encoded by the nucleotide sequence of SEQ ID NO: 2.

As used herein, the term “malonyl-CoA-[acyl-carrier protein] transacylase” refers to the enzyme that catalyses the conversion of malonyl-CoA to malonyl-ACP and is encoded by the fabD gene.

Preferably, the malonyl-CoA-[acyl-carrier protein] transacylase expressed from the expression vector according to the present invention is derived from Pseudomonas aeruginosa. More preferably, the malonyl-CoA-[acyl-carrier protein] transacylase has the amino acid sequence of SEQ ID NO: 3, encoded by the nucleotide sequence of SEQ ID NO: 4.

As used herein, the term “acyl-acyl carrier protein thioesterase” refers to the enzyme which is indispensible for the biological production of long-chain fatty acids on the fatty acid biosynthesis pathway and is involved in the extension of fatty acid chains from malonyl-CoA. This enzyme is encoded by the 3.1.2.14 gene and is not found in wild-type E. coli.

The acyl-acyl carrier protein thioesterase expressed from the expression vector according to the present invention is preferably Streptococcus derived. More preferably, the acyl-acyl carrier protein thioesterase has the amino acid sequence of SEQ ID NO: 5, encoded by the nucleotide sequence of SEQ ID NO: 6 isolated from Streptococcus pyogenes.

It should be understood to those skilled in the art that nucleotide sequences useful in the present invention are not limited to the above-mentioned nucleotide sequences, but may include nucleotide sequences with a substantial identity with the above-mentioned nucleotide sequences. The substantial identity is at least 80%, more preferably at least 90%, and most preferably at least 95% as analyzed by typically used algorithm from most probable alignments of nucleotide sequences. Alignment methods for sequence comparison are well known in the art. With regard to various alignment methods and algorithms, reference may be made to Smith and Waterman, Adv. Appl. Math. 2:482 (1981); Needleman and Wunsch, J. Mol. Bio. 48:443 (1970); Pearson and Lipman, Methods in Mol. Biol. 24: 307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992) and Pearson et al., Meth. Mol. Biol. 24:307-31 (1994). The Basic Local Alignment Search Tool (BLAST) program (Altschul et al., J. Mol. Biol. 215:403-10 (1990)), developed by the U.S. National Center for Biological Information (NCBI), can be accessed for free over the web http://www.ncbi.nlm.nih.gov/BLAST/, hosted by the NCBI, and can be used in association with a sequence analysis program, such as blastp, blasm, blastx, tblastn and tblastx, on the web. The website http://www.ncbi.nlm.nih.gov/BLAST/_help.html teaches how to use the BLAST program to perform sequence similarity against the database.

The reaction mechanisms for which the above-mentioned genes are responsible are summarized as follows.

accA gene: ATP+acetyl-CoA+HCO3-<=>ADP+orthophosphage+malonyl-CoA,

fabD gene: malonyl-CoA+acyl-carrier protein<=>CoA+malonyl[acyl-carrier protein],

E.3.1.2.14 gene: oleoyl-[acyl-carrier protein]+H(₂)O<=>[acyl-carrier protein]+oleate.

After being inserted into an expression vector, these genes are expressed in E. coli. As used herein, the term “expression vector” refers to a linear or circular DNA molecule in which a gene encoding a polypeptide of interest is operatively linked to a regulatory element for transcription. The regulatory element comprises a promoter and a termination codon. In addition, the expression vector contains at least one replication origin, at least one selection marker, and a polyadenylation signal. Also, it may be derived from either a plasmid or a viral DNA, or may contain both.

The vector system of the present invention may be constructed using various methods well known in the art, and details thereof are described in Sambrook et al., Molecular Cloning, A Laboratory Manual, Cold Spring Harbor Laboratory Press (2001), which is herein incorporated by reference.

The nucleic acids encoding the enzymes of the present invention are operatively linked to a promoter which is operated in eukaryotic cells. As used herein, the term “operatively linked” refers to the functional association of nucleic acid sequences on a single regulatory sequence (e.g., a promoter, a signal sequence or an array of transcriptional regulatory factor binding sites) so that the transcription and/or translation of the nucleic acid sequences is regulated by the regulatory sequence.

The vector of the present invention may be constructed to provide typical expression. When the vector of the present invention is a prokaryotic expression vector, it may typically contain a potent promoter (e.g., tac promoter, lac promoter, lacUV5 promoter, lpp promoter, pL^{λ} promoter, pR^{λ} promoter, rac5 promoter, amp promoter, recA promoter, SP6 promoter, trp promoter and T7 promoter), a ribosomal binding site for initiating translation, and transcription/translation terminating sequences. When E. coli (e.g., HB101, BL21, DH5α, etc.) is used as a host cell, a promoter and operator for E. coli tryptophan biosynthesis pathway (Yanofsky, C., J. Bacteriol., 158:1018-1024 (1984)) and a phage λ left promoter (pL^{λ} promoter, Herskowitz, I. and Hagen, D., Ann. Rev. Genet., 14:399-445 (1980)) may be recruited as regulatory factors.

A vector available for the present invention may be engineered from typically used plasmids (e.g., pSC101, pGV1106, pACYC177, ColE1, pKT230, pME290, pBR322, pUC8/9, pUC6, pBD9, pHC79, pIJ61, pLAFR1, pHV14, pGEX series, pET series and pUCP19), phage (e.g., λ-Charon and M13) or virus (e.g., SV40, etc.), and preferably may be pUCP19, which can deliver exogenous genes of interest into the host cell E. coli and can effectively regulate the expression of the exogenous genes in the E. coli.

Also, the vector of the present invention contains a selection marker which is typically an antibiotic resistance gene, for example, a gene for resistance to ampicillin, gentamicin, carbenicillin, chloramphenicol, streptomycin, kanamycin, geneticin, neomycin, or tetracycline.

The expression vector of the present invention comprises a promoter sequence, a nucleotide sequence of a gene of expression interest (structural gene), and a terminator sequence, which are connected sequentially in the direction of 5′→3′. In the present invention, at least one gene selected from among accA and fabD, and the E3.1.2.14 gene are linked sequentially in the 5′→3′ direction.

In order for the genes to have minimal lengths in the expression vector of the present invention, nucleotide sequences comprising only the elements necessary for enzyme expression, such as RBS (ribosomal binding site), etc., are preferably inserted into the expression vector in light of alleviating a metabolic burden to the host cell.

The expression vector carrying the genes is introduced into E. coli by a transformation method. Typical among the transformation method are a CaCl₂ method (Cohen, S. N. et al., Proc. Natl. Acad. Sci. USA, 9:2110-2114 (1973)), a Hanahan method (Cohen, S. N. et al., Proc. Natl. Acad. Sci. USA, 9:2110-2114 (1973); and Hanahan, D., J. Mol. Biol., 166:557-580 (1983)) and electroporation (Dower, W. J. et al., Nucleic. Acids Res., 16:6127-6145 (1988)). Preferred is electroporation in terms of the stable and efficient construction of transformants.

In accordance with another aspect thereof, the present invention addresses a method for preparing an E. coli transformant for overexpressing a fatty acid, comprising: (a) inserting into an expression vector at least one nucleotide sequence selected from the group consisting of a nucleotide sequence coding for acetyl-CoA carboxylase carboxytransferase subunit alpha, a nucleotide sequence coding for malonyl-CoA-[acyl-carrier protein] transacylase, and a nucleotide sequence coding for acyl-acyl carrier protein thioesterase; and (b) transforming E. coli with the expression vector carrying the nucleotide sequence.

Because the method for preparing E. coli for overexpressing an enzyme involved in the fatty acid biosynthesis pathway is concerned with the preparation of the E. coli transformant, a description of the common content therebetween has been omitted to avoid making the specification excessively complex.

In accordance with a further aspect thereof, the present invention addresses a method for the biosynthesis of a fatty acid, comprising: (a) culturing the E. coli transformant to allow a fatty acid to be synthesized; and (b) recovering the fatty acid.

The E. coli transformant for producing a fatty acid in accordance with the present invention may be cultured using a typical method well known in the art.

Preferably, the culturing step (a) is carried out in the presence of the expression inducer IPTG.

The recovery of the fatty acid synthesized in the E. coli transformant may be implemented using typical separation or purification well known in the art (refer to B. Aurousseau et al., Journal of the American Oil Chemists' Society, 57(3):1558-9331 (1980); Frank C. Magne et al., Journal of the American Oil Chemists' Society, 34(3):127-129 (1957)).

In accordance with still a further aspect thereof, the present invention addresses the use of the E. coli transformant in the biosynthesis of a fatty acid.

Expressing an enzyme involved in the fatty acid biosynthesis pathway, the E. coli transformant according to the present invention produces a fatty acid in high yield and thus can be used for the mass production of a fatty acid.

### Advantageous Effects

Features and advantages of the present invention are summarized as follows:

(a) The present invention provides E. coli, capable of producing a fatty acid in high yield, transformed with an expression vector carrying: at least one a nucleotide sequence selected from the group consisting of a nucleotide sequence coding for acetyl-CoA carboxylase carboxytransferase subunit alpha, a nucleotide sequence coding for malonyl-CoA-[acyl-carrier protein] transacylase and a nucleotide sequence coding for acyl-acyl carrier protein thioesterase.

(b) The present invention can not only promote the production of malonic acid, a metabolite in an early phase of the fatty acid biosynthesis pathway, in the recombinant heterologous E. coli, but can increase the content of fatty acid itself within the E. coli.

(c) The present invention allows enzymes involved in the fatty acid biosynthesis from glucose to be overexpressed and modifies the metabolism flow by introducing exogenous genes, thus producing a fatty acid in high yield.

(d) The present invention is anticipated to be used for the mass production of fatty acids, useful as a bioenergy source, in an environmentally friendly and economically beneficial manner.

### DESCRIPTION OF DRAWINGS

FIG. 1 is a schematic view of the fatty acid biosynthesis pathway ranging from glucose to fatty acids.

FIG. 2 is a schematic view of the genetic map of the Escherichia coli-Pseudomonas shuttle vector pUCP19.

FIG. 3 is a schematic view of the genetic map of the Escherichia coli-Pseudomonas shuttle vector pUCP19 transformed with the 3.1.2.14 gene, named pJS04.

FIG. 4 is a schematic view of the genetic map of the Escherichia coli-Pseudomonas shuttle vector pUCP19 transformed with accA, fabD and 3.1.2.14 genes, named pJS07.

FIG. 5 is a photograph after the recombinant plasmids of FIGS. 3 and 4, carrying accA and fabD genes of Pseudomonas aeruginosa, and/or the 3.1.2.14 enzyme gene of Streptococcus pyogenes, were treated with restriction enzymes and electrophoresed. Lane 1: size marker, lanes 2-4: the 3.1.2.14 gene digested from E. coli SGJS14 with SacI and EcoRI, lane 5: blank, lanes 6 and 7: the 3.1.2.14 gene digested from E. coli SGJS17 with SacI and EcoRI, lane 8: the 3.1.2.14 gene digested from E. coli SGJS17 with XbaI and BamHI, lane 9: fabD gene digested from E. coli SGJS17 with KpnI and SacI.

FIG. 6 is a graph showing the growth of the recombinant strains E. coli SGJS04, E. coli SGJS05, E. coli SGJS06, E. coli SGJS14 and E. coli SGJS17, and the wild-type.

FIG. 7 shows extracellular levels of acetic acid and malonic acid secreted from the recombinant E. coli strains of the present invention and the wild-type.

FIG. 8 shows intracellular levels of lipids extracted from the recombinant E. coli strains of the present invention and the wild-type after culture for 24 hrs.

FIG. 9 is a GC spectrum showing the production of the fatty acids hexadecanoic acid, 9-hexadecanoic acid, 7-hexadecanoic acid, heptadecanoic acid, 9-octadecanoic acid, and octadecanoic acid from the recombinant E. coli strain of the present invention.

FIG. 10 shows GC/MS spectra of C-16 hexadecanoic acid.

FIG. 11 shows GC/MS spectra of C-18 octadecanoic acid.

FIG. 12 shows levels of hexadecanoic acid and octadecanoic acid in the recombinant E. coli strains of the present invention and the wild-type after IPTG induction for 6 and 24 hrs.

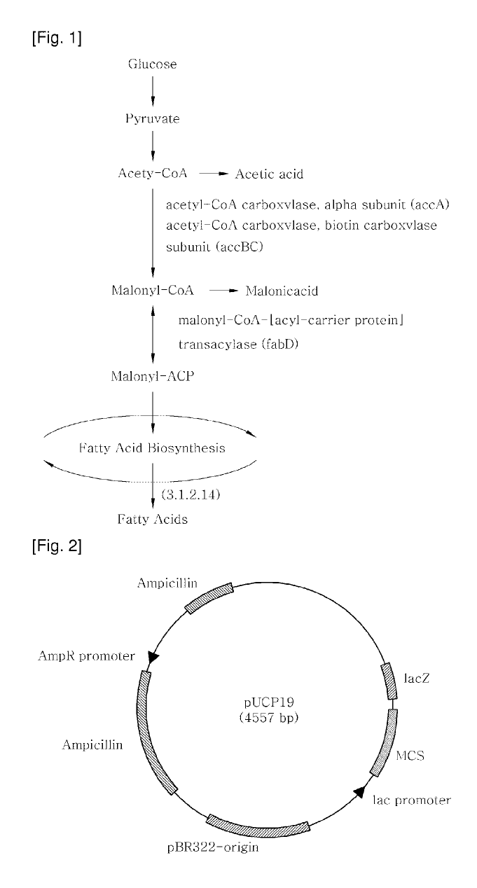

### MODE FOR INVENTION

A better understanding of the present invention may be obtained through the following examples which are set forth to illustrate, but are not to be construed as limiting the present invention.

### Example 1

### Construction of Recombinant Plasmids Carrying accA and fabD Genes and Carrying 3.1.2.14 Gene Using Polymerase Chain Reaction (PCR)

Nucleotide sequences of accA and fabD (GenBank, NCBI), which encode enzymes responsible for the production of malonyl-CoA and malonyl-CoA:ACP, respectively, were amplified by PCR (DaKaRa, Korea) using primers with the genomic DNA of Pseudomonas aeruginosa PAO1 (KCCM) serving as a template. Separately, PCR was performed on the genomic DNA of Streptococcus pyogenes MGAS10270 (ATCC) in the presence of primers suitable for amplifying a gene encoding acyl-acyl carrier protein thioesterase (E.C 3.1.2.14) (GenBank, NCBI), which is new to both E. coli and Pseudomonas aeruginosa (Tables 1 and 2).

TABLE 1

| Gene | Enzyme |
|---|---|
| accA | acetyl-CoA carboxylase, carboxytransferase, |
| | alpha subunit |
| fabD | malonyl-CoA-[acyl-carrier-protein] transacylase |
| 3.1.2.14 | acyl-acyl carrier protein thioesterase |

TABLE 2
Primers for the Amplification of Genes of
Table 1, with Restriction Sites

| | | | Restric- |
|---|---|---|---|
| | | | tion |
| Gene | Primer Sequence | | Enzyme |
| accA | F | 5′-TCTAGACGACGGAAGCCTATGAA | XbaI |
| | | CCCG-3′ | |
| accA | R | 5′-GGATCCTTACGGCGCGCCGTAGC | BamHI |
| | | TCAT-3′ | |
| fabD | F | 5′-GGTACCCAAGGGACCTATTCAAT | KpnI |
| | | GTCTGC-3′ | |
| fabD | R | 5′-GAGCTCTTCTCTCCTTTCTCTCT | SacI |
| | | CTCAGG-3′ | |
| E.C | F | 5′-GAGCTCGGAGAGTATTATGGGAT | SacI |
| 3.1.2.14 | | TAAGTTA-3′ | |
| E.C | R | 5′-GAATTCCTAGTCTATCTCGCTTT | EcoRI |
| 3.1.2.14 | | CTGTTT-3′ | |

The genes of Table 1 were amplified using the primers of Table 2 which were designed to specifically target the genes of interest. Each of these genes contained an RBS (ribosomal binding site) and amplification was completed at 972 bp for accA, at 963 bp for fabD, and at 763 bp for the gene coding for acyl (acyl carrier protein) thioesterase. After starting with one cycle of 95° C./5 min (denaturation), 66° C./1 min (annealing), and 72° C./1 min (extension) under a typical condition (10 mM Tris-HCl (pH9.0), 50 mM KCl, 0.1% Triton X-100, 2 mM MgSO₄, Taq DNA polymerase (DaKaRa)), each PCR was performed with 30 cycles of 95° C./1 min (denaturation), 66° C./30 sec (annealing), and 72° C./1 min (extension). For stable extension, a final cycle of 95° C./1 min (denaturation), 66° C./1 min (annealing), and 72° C./5 min (extension) was carried out. The PCR products thus obtained were identified on 0.8% agarose gel and purified before use in T-vector cloning. The genes of interest were ligated to pGEM-T easy vector (DaKaRa) to construct recombinant plasmids pGEM-T:accA, pGEM-T:fabD, and pGEM-T::3.1.2.14. The recombinant plasmids were transformed into E. coli (AG1 competent cell, Stratagene) to prepare novel species.

### Example 2

### Construction of Recombinant Plasmids pJS04 and pJS07

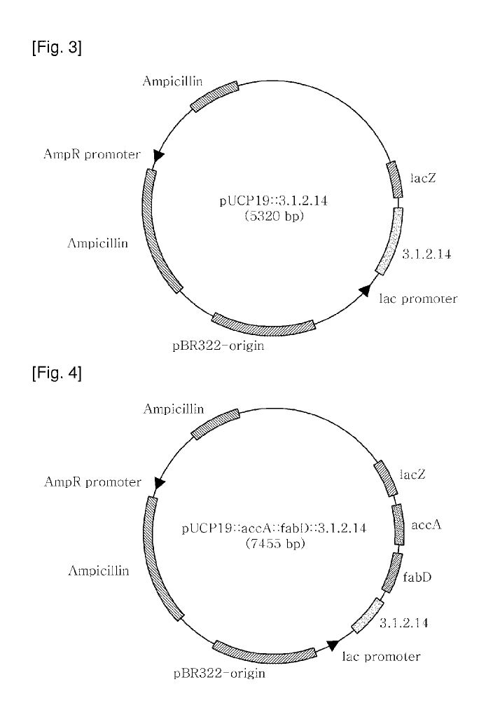

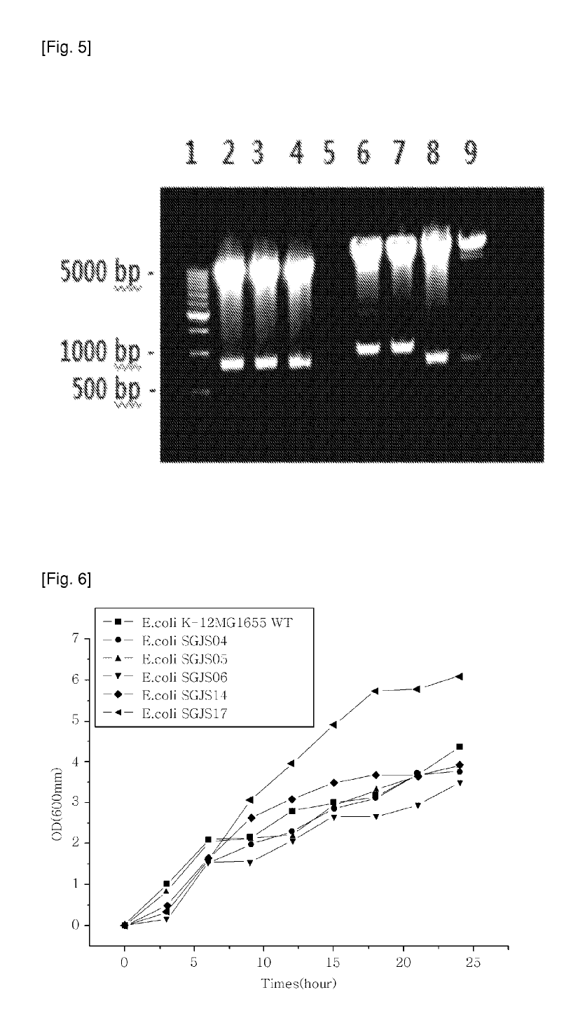

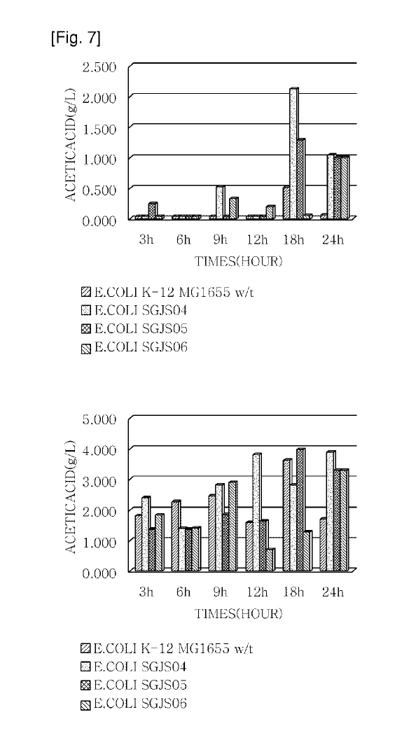

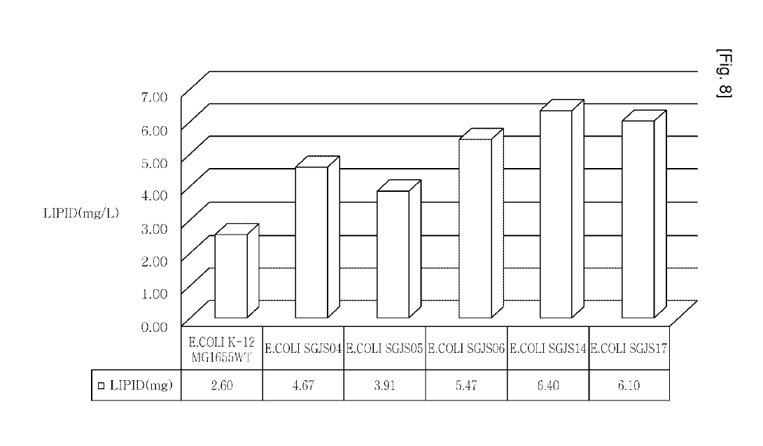

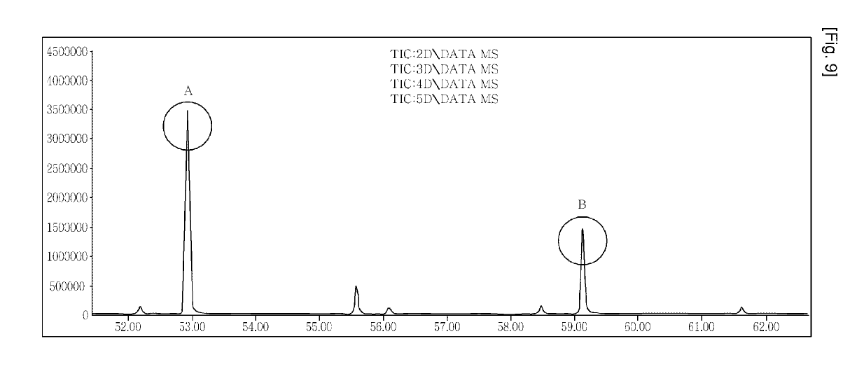

pGEM-T::3.1.2.14, pGEM-T:accA, and pGEM-T:fabD, recombinant plasmids of Example 1, and pUCP19 (ATCC, FIG. 2), a shuttle vector for both E. coli and Pseudomonas, were separately treated for 2 hrs with the restriction enzymes of Table 2 in a 37° C. water bath. The resulting DNA digests were inserted into the multi-cloning site of pUCP19, as shown in FIGS. 3 and 4, by ligation at 16° C. using T4 ligase (Dakara). The cloning of each of the genes was examined every insertion. After transformation into E. coli (AG1 competent cell, Stratagene), the recombinant plasmid was isolated and digested with the same restriction enzymes as were used for insertion, and the size of the DNA digest was determined on 0.8% agarose gel by electrophoresis. FIG. 5 shows inserts digested from the recombinant plasmids by restriction enzymes. After digestion with the restriction enzymes of Table 2, the PCR products were introduced into pUCP19 to afford recombinant plasmids pJS01, pJS02, pJS03, pJS04, and pJS07. FIGS. 3 and 4 are genetic maps of the vectors pJS04 (carrying the 3.1.2.14 gene), and pJS07 (carrying accA, fabD and 3.1.2.14 genes).

### Example 3

### Preparation of Heterologous E. coli Transformant

For use in transformation, E. coli is typically made competent using CaCl₂ buffer before introduction of a plasmid by heat shock. In the present invention, electroporation was used in consideration of the stability and efficiency of transformation. Before electroporation, wild-type Escherichia coli K-12 MG1655 (ATCC, USA) was pre-cultured for 16 hrs and 30 μl (0.1%) of the culture was inoculated into 3 mL of LB (10 g/L tryptone, 10 g/L NaCl, 5 g/L yeast extract) and centrifuged to separate cells (12,000 rpm, 1 min) and the medium when absorbance at 600 nm reached 0.6. Then, the cells were washed once with 1 mL of 10% glycerol and harvested by centrifugation (12,000 rpm, 1 min). The cell pellet was resuspended in 80 μL of 10% glycerol. To the suspension was added 1-3 μL of the recombinant plasmids (pJS01, pJS02, pJS03, pJS04, and pJS07). The mixtures, each amounting to greater than 80 μl, were placed in respective cuvettes for electroporation (BIO-RAD, Gene pulser cuvette) and electrically shocked using BIO-RAD, Gene pulser Xcell (1800 v, 25 μF, 200Ω). Immediately after the electric shock, each of the mixtures was mixed with 1 mL of LB (10 g/L tryptone, 10 g/L NaCl, 5 g/L yeast extract) and incubated at 37° C. 1 hour with agitation at 200 rpm. The cells were cultured at 37° C. on LB agar plates (10 g/L tryptone, 10 g/L NaCl, 5 g/L yeast extract, agar 20 g/L) containing amphicillin (50 μg/mL) to form single colonies. Thus, the recombinant strains E. coli SGJS04, E. coli SGJS05, E. coli SGJS06, E. coli SGJS14, and E. coli SGJS17 resulting from the transformation of Escherichia coli K-12 MG1655 with the recombinant plasmids pJS01, pJS02, pJS03, pJS04, and pJS07, respectively, were obtained (Table 3).

TABLE 3
Recombinant Strains

| Strain | Genotype |
|---|---|
| E. coli SGJS04 | E. coli K-12 MG1655::pUCP19::accA |
| E. coli SGJS05 | E. coli K-12 MG1655::pUCP19::fabD |
| E. coli SGJS06 | E. coli K-12 MG1655::pUCP19::accA::fabD |
| E. coli SGJS14 | E. coli K-12 MG1655::pUCP19::3.1.2.14 |
| E. coli SGJS17 | E. coli K-12 |
| | MG1655::pUCP19::accA::fabD::3.1.2.14 |

### Example 4

### Culturing of Heterologous E coli Strain and Protein Production Therefrom

Recombinant strains of Escherichia coli K-12 MG1655 were pre-cultured for 16 hrs in an LB broth (containing 50 μg/mL ampicillin). Then, the culture was inoculated into an LB broth containing 50 μg/mL ampicillin and cultured until absorbance at 600 nm reached 0.6-1.0. At this time, the bacterial culture was mixed with a final concentration of 25% glycerol to afford stocks which were stored at −80° C. until use in subsequent experiments. After being thawed, 30 μL from the stock was cultured in 3 mL of LB containing ampicillin (50 μg/mL) in a 10 mL bottom tube. Of the culture, 1 mL was inoculated into 200 mL of LB (10 g/L tryptone, 10 g/L NaCl, 5 g/L yeast extract) in a 500 mL flask and cultured for 24 hrs at 37° C. with agitation at 170 rpm. For comparison, wild-type Escherichia coli K-12 MG1655 was cultured under the same conditions as in the recombinant strains with the exception that the LB broth was free of ampicillin. When absorbance at 600 nm of the cultures reached 0.6, protein expression was induced by IPTG (isopropylthio-β-D-galactoside, sigma, USA) (final concentration of 1 mM/mL). Growth curves of the recombinant E. coli strains designed to increase the production of fatty acids by overexpressing enzymes involved in the fatty acid biosynthesis pathway, and the wild-type are given in FIG. 6.

As shown in the curves of FIG. 6, there were no great differences in growth between the heterologous recombinant E. coli strains and the wild-type, indicating that the introduction or overexpression of exogenous genes exerted neither toxicity nor inhibition. This is thought to be attributable to the fact that the vector was able to allow stable expression in both Pseudomona and E. coli and each gene of interest contained an RBS (ribosomal binding site) to provide for expression. In this context, the cording region of each gene was designed to have the minimal length required of a nucleotide sequence comprising only indispensible elements, such as RBS and an overexpression function, so that the metabolic burden placed on the host cell was reduced. In addition, the two genes were arranged in the order of their reactions in the biosynthesis pathway.

### Example 5

### Extracellular Level of Acetic Acid and Malonic Acid from the Recombinant E. coli Strains

Extracellular levels of acetic acid and malonic acid, which are intermediates in the fatty acid biosynthesis pathway and act as inducers of fatty acid biosynthesis, were quantitatively analyzed. Samples were taken at regular intervals during cell culturing and centrifuged (12000 rpm, 10 min). The supernatants were stored at −40° C. before analysis. Of each of the supernatants, 1 mL was filtered through a 0.2 μm filter and quantitatively analyzed using HPLC under the following conditions.

TABLE 4
HPLC Conditions for Quantitative Analysis of
Acetic Acid and Malonic Acid

| | Condition |
|---|---|
| Detector | UV730D (Younglin Instrument, Korea) |
| Column | Aminex HPX-87H (Biorad, USA) |
| Flow Rate | 0.6 mi/min |
| Injection Volume | 30 μL |
| Mobile Phase | 0.005N Sulfuric acid |
| Oven Temp. | 50° C. |
| Working Time | 30 min |
| UV Detection | 210 nm |

Both acetic acid and malonic acid are produced as intermediates in an early stage of the fatty acid biosynthesis pathway and act as inducers of fatty acid biosynthesis. In this regard, extracellular levels of acetic acid and malonic acid in the cultures of the three recombinant strains E. coli SGJS04, E. coli SGJS05 and E. coli SGJS06, and the wild-type E. coli were monitored. There was a difference in the tendency to produce acetic acid and malonic acid among the recombinant strains as well as the wild-type E. coli. At a relatively short time after culturing, a higher level of malonic acid was detected in the culture of E. coli SGJS04 with the accA gene than the cultures of the other recombinant stains and the wild-type. E. coli SGJS05 and E. coli SGJS06, both having fabD, were observed to produce malonic acid in smaller amounts than did E. coli SGJS04 with accA.

The abundant malonic acid in E. coli SGJS04 could be readily accepted from the inference that acetyl-CoA would be actively converted into malonyl-CoA and then to malonic acid as the accA gene encoding acetyl-CoA carboxylase was overexpressed. The overexpression of fabD in E. coli SGJS05 and E. coli SGJS06 increase the activity of malonyl-CoA[acyl carrier protein] transacylase to promote the entry of malonyl-CoA through malonyl-acyl carrier protein into the elongation phase of fatty acid biosynthesis, which results in the depletion of malonyl-CoA before conversion to malonic acid. Accordingly, these recombinant stains produced lower levels of malonic acid than did E. coli SGJS04. That is, the overexpression of enzymes which play critical roles in early stages of the fatty acid biosynthesis pathway increased the level of malonic acid, an intermediate in fatty acid biosynthesis, which would be expected to bring about an improvement in lipid content.

### Example 6

### Intracellular Lipid Levels of Wild-Type E. coli and Recombinant Strains

Lipids were extracted using a modified version of the Bligh-Dyer method (1959). After incubation for 24 hrs in the presence of IPTG under the same conditions as in Example 4, 50 mL of each of the cultures was centrifuged (4500 rpm for 10 min). The cell pellet was suspended in PBS (phosphate-buffer saline, 50 mM, pH 7.4) and centrifuged. The cells of the recombinant strains and the wild-type were vortexed in 2 mL of methanol (MeOH) and then vortexed again together with 1 mL of chloroform (CHCl₃). Then, 0.8 mL of sterile distilled water was added to the solution and sufficiently vortexed. Again, this resulting mixture was vigorously mixed with 1 mL of chloroform by vortexing. After centrifugation (1000 rpm for 20 min), the upper layer was withdrawn, transferred into a new tube and dried before carrying out a quantitative analysis of the intracellular lipid levels.

All of the recombinant E. coli strains of the present invention were observed to have increased lipid content, compared to the wild-type, as can be seen in FIG. 8. Because lipids contain fatty acids, an increased fatty acid content was thought to lead to an increase in lipid content. The lipid content was found to increase to a relatively higher extent in E. coli SGJS04 and E. coli SGJS06, both transformed with the accA gene of Pseudomonas aeruginosa and to a relatively lesser extent in E. coli SGJS05 with the fabD gene of Pseudomonas aeruginosa, indicating that the fabD gene of Pseudomonas aeruginosa is slightly inhibited in the production of fatty acids, compared to the accA gene. The highest lipid content was detected in E. coli SGJS14 with the 3.1.2.14 gene of Streptococcus pyogenes. Thus, the 3.1.2.14 gene was identified as playing an important role in the biosynthesis of fatty acids. E. coli SGJS17 in which the genes of Pseudomonas aeruginosa and Streptococcus pyogenes were co-expressed produced a slightly smaller level of lipids than did the strain with only the gene of Streptococcus pyogenes, which is thought to result from the co-expression of heterogeneous genes.

### Example 7

### Intracellular Fatty Acid Levels of the Wild-Type E. coli and the Recombinant Strains

When sufficient amounts of the cells were obtained 6 and 24 hrs after the incubation in the presence of IPTG under the same conditions as those of Example 4, the extraction of fatty acid from the cells was carried out in the following five steps. The first step was cell harvesting. Of the culture, 5 mL was centrifuged (4500 rpm, 10 min) and the cells thus harvested were stored at −80° C. The next step was saponification. The cells were vortexed for 5-10 sec in 1 mL of Solution 1 (NaOH 45 g, MeOH 150 mL, deionized distilled water 150 mL). After reaction at 100° C. for 5 min, the cells were again vortexed for 5-10 sec. The reaction mixture was maintained at 100° C. for 25 min and then cooled. Thereafter, methylation was carried out. The reaction mixture was mixed vigorously with 2 mL of Solution 2 (6N HCl 325 mL, MeOH 275 mL) by vortexing for 5-10 sec, followed by reacting at 80° C. for 10 min. Immediately after the reaction, the temperature was reduced. Extraction was the fourth step. The resulting mixture was mixed with 1.25 mL of Solution 3 (Hexane/Methyl tert-Butyl Ester=1/1), followed by shaking up and down for 10 min. The lower layer that was formed thereby was discarded. Finally, washing was performed to facilitate GC analysis. The remaining layer was mixed with 3 mL of Solution 4 (NaOH 10.8 g, ddW 900 mL) and shaken up and down for 5 min. The upper layer thus formed was withdrawn and subjected to GC/MS analysis.

For CG/MS analysis, Agilent 7890A gas chromatography with 5975 series mass-selective detector (MSD) was equipped with HP-5 column (30 m×0.32 mm, film thickness 0.25). Helium gas was employed as a mobile phase. For quantitative analysis, samples were run against a fatty acid methyl ester standard (F.A.M.E Mix, C8-C24, Sigma). GC conditions followed the temperature program of: 40° C. 5 min, to 220° C. at a rate of 3° C./min, to 250° C. at a rate of 3° C. per min, and 250° C. 5 min, and the internal temperature of MS was 160° C.

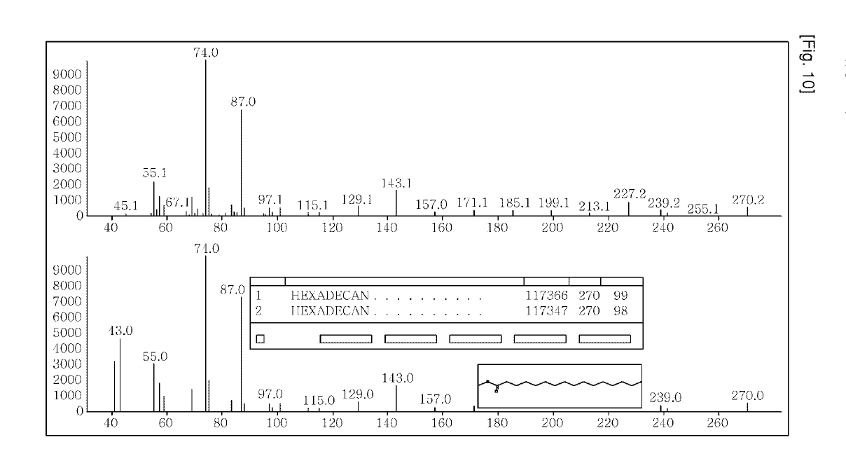

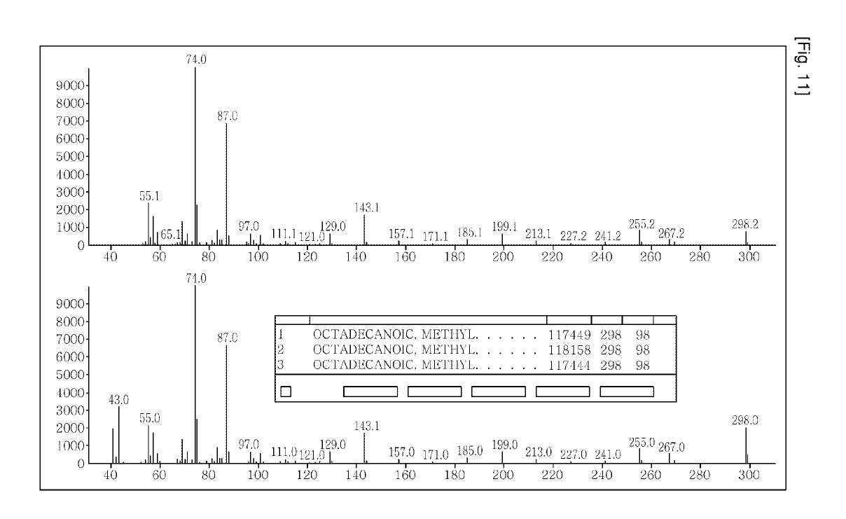

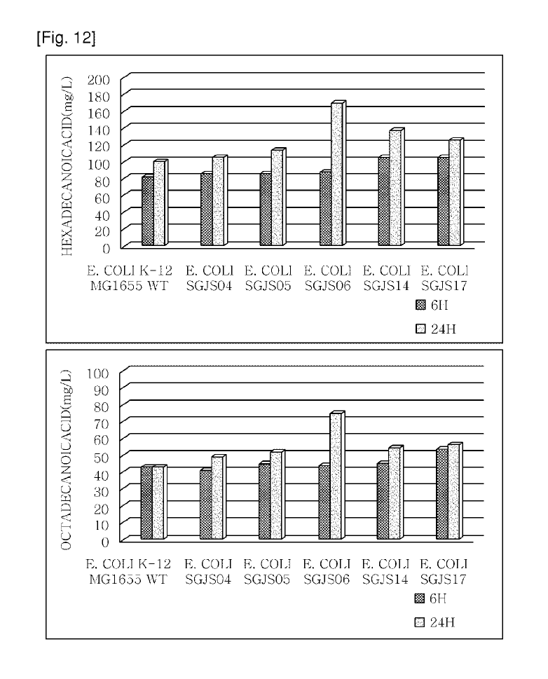

Analysis of the fatty acids extracted from two transformed recombinant E. coli strains and the wild-type is shown in FIGS. 9 to 12. In FIG. 9, various kinds of the fatty acids that were extracted were identified, including hexadecanoic acid (A). In addition, also identified were octadecanoic acid (B), 9-hexadecanoic acid, 7-hexadecanoic acid, heptadecanoic acid and 9-octadecanoic acid. The predominant fatty acids of hexadecanoic acid (A) and octadecanoic acid (B) were further quantitatively analyzed against the standard, and the results are given in FIG. 12. Hexadecanoic acid contains 16 carbon atoms while octadecanoic acid is a C-18 fatty acid. FIG. 10 shows levels of the fatty acids extracted 6 and 24 hrs after IPTG induction.

As can be seen from the data, the heterologous recombinant E. coli strains produced higher levels of both hexadecanoic acid and octadecanoic acid than did the wild-type. While levels of the fatty acids produced by E. coli SGJS04, E. coli SGJS05, and E. coli SGJS06, in all of which the Pseudomonas aeruginosa gene or genes involved in the early stage of the fatty acid biosynthesis pathway were cloned, were similar to those in the wild-type 6 hrs after IPTG induction, E. coli SGJS14 and E. coli SGJS17 in both of which acy (acyl carrier protein) thioesterase of Streptococcus pyogenes was expressed were found to increase in both hexadecanoic acid and octadecanoic acid levels, compared to the wild-type. When the cells were cultured for a long period of time, e.g., 24 hrs, the amounts of the fatty acids were increased even for E. coli SGJS14 and E. coli SGJS17, and peaked in E. coli SGJS06.

1. An Escherichia coli strain, capable of producing a fatty acid in high yield, being transformed with an expression vector carrying at least one gene selected from the group consisting of:
(a) a nucleotide sequence coding for acetyl-CoA carboxylase carboxytransferase subunit alpha of Pseudomonas aeruginosa,
(b) a nucleotide sequence coding for malonyl-CoA-[acyl-carrier protein] transacylase of Pseudomonas aeruginosa, and
(c) a nucleotide sequence coding for acyl-acyl carrier protein thioesterase of Streptococcus pyogenes and a combination thereof.

2. The Escherichia coli strain of claim 1, being transformed with an expression vector carrying
(a) a nucleotide sequence coding for acetyl-CoA carboxylase carboxytransferase subunit alpha of Pseudomonas aeruginosa, and
(b) a nucleotide sequence coding for malonyl-CoA-[acyl-carrier protein] transacylase of Pseudomonas aeruginosa, and

3. The Escherichia coli strain of claim 1, being transformed with an expression vector carrying a nucleotide sequence coding for acyl-acyl carrier protein thioesterase of Streptococcus pyogenes.

4. The Escherichia coli strain of claim 1, being transformed with an expression vector carrying:
(a) a nucleotide sequence coding for acetyl-CoA carboxylase carboxytransferase subunit alpha of Pseudomonas aeruginosa,
(b) a nucleotide sequence coding for malonyl-CoA-[acyl-carrier protein] transacylase of Pseudomonas aeruginosa, and
(c) a nucleotide sequence coding for acyl-acyl carrier protein thioesterase of Streptococcus pyogenes and a combination thereof.

5. The Escherichia coli strain of claim 1, wherein the expression vector is an E. coli-Pseudomonas shuttle vector having a genetic map of FIG. 2, identified as pUCP19, and the nucleotide sequences are inserted into MCS (multiple cloning site) of pUCP19.

6. The Escherichia coli strain of claim 1, wherein the acetyl-CoA carboxylase carboxytransferase subunit alpha the malonyl-CoA-[acyl-carrier protein] transacylase, and the acyl-acyl carrier protein thioesterase have amino acid sequences of SEQ ID NOS: 1, 3, and 5, respectively.

7. The Escherichia coli strain of claim 6, wherein the acetyl-CoA carboxylase carboxytransferase subunit alpha, the malonyl-CoA-[acyl-carrier protein] transacylase, and the acyl-acyl carrier protein thioesterase nucleotide are encoded by nucleotide sequences of SEQ ID NOS: 2, 4, and 6, respectively.

8. A method for preparing a recombinant Escherichia coli strain capable of producing a fatty acid in high yield, comprising:
(a) inserting into an expression vector a gene selected from the group consisting of:
i) a nucleotide sequence coding for acetyl-CoA carboxylase carboxytransferase subunit alpha of Pseudomonas aeruginosa,
ii) a nucleotide sequence coding for malonyl-CoA-[acyl-carrier protein] transacylase of Pseudomonas aeruginosa, and
iii) a nucleotide sequence coding for acyl-acyl carrier protein thioesterase of Streptococcus pyogenes and a combination thereof, and
(b) transforming the expression carrying the nucleotide into Escherichia coli.

9. The method of claim 8, wherein the transforming step (b) is carried out by electroporation.

10. A method for biosynthesis of a fatty acid, comprising:
(a) culturing the Escherichia coli strain of claim 1 to produce the fatty acid; and
(b) recovering the fatty acid of step (a) from the cell culture.

11. Use of the Escherichia coli of claim 1 in biosynthesis of a fatty acid.
